(19) United States
(12) Patent Application Publication
(10) Pub. No.: US 2010/0247445 A1
(43) Pub. Date: Sep. 30, 2010

(54) POLYMERIC DRUG CARRIER FOR IMAGE-GUIDED DELIVERY

(75) Inventors: Sander Langereis, Eindhoven (NL); Holger Gruell, Eindhoven (NL); Lea Louise Pauline Messager, Morlaix (FR); Jeroen Alphons Pikkemaat, Eindhoven (NL); Dirk Burdinski, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/746,204

(22) PCT Filed: Dec. 4, 2008
(86) PCT No.: PCT/IB08/55098

§ 371 Date: Jun. 4, 2010

(30) Foreign Application Priority Data
Dec. 7, 2007 (EP) 07122621.1
Mar. 25, 2008 (EP) 08153219.4

(51) Int. Cl. A61B 5/055; A61P 35/00

(52) U.S. Cl. 424/9.3

(57) ABSTRACT

Described are Chemical Exchange-dependent Saturation Transfer (CEST) contrast agents for Magnetic Resonance Imaging (MRI) comprising a polymersome provided with a paramagnetic agent. The polymersome preferably comprises a polymeric shell enclosing a cavity, wherein the cavity comprises a pool of proton analytes, and wherein the shell allows diffusion of the proton analytes. The polymersome-based CEST MRI contrast agents are suitable as drug carriers useful in MRI-guided drug release.

### FIELD OF THE INVENTION

The invention relates to image guided drug delivery based on polymersomes as drug carriers. More particularly, the invention relates to the Magnetic Resonance Imaging (MRI) monitored or guided delivery of bio-active agents such as therapeutic or diagnostic agents (hereinafter referred to as “drugs”). Furthermore, the invention pertains to Chemical Exchange-dependent Saturation Transfer (CEST) contrast agents for Magnetic Resonance Imaging (MRI), particularly to agents that are suitable for use as drug carriers.

### BACKGROUND OF THE INVENTION

Many diseases that are mostly localized in a certain tissue are treated with systemically administered drugs. A well-known example of standard cancer therapy is a systemic chemotherapy coming along with significant side effects for the patient due to undesired biodistribution and toxicity. The therapeutic window of these drugs is usually defined by the minimal required therapeutic concentration in the diseased tissue on the one hand, and the toxic effects in non-targeted organs, e.g. liver, spleen, on the other. Localized treatment by, for example, local release of cytostatics from nanocarriers promises a more efficient treatment and a larger therapeutic window compared to standard therapeutics. Localized drug delivery is also important if other therapeutic options such as surgery are too risky as is often the case for liver cancers. Localized drug delivery can also become the preferred treatment option for many indications in cardiovascular disease (CVD), such as atherosclerosis in the coronary arteries.

Magnetic Resonance Imaging is an important diagnostic technique that is commonly used in hospitals for the diagnosis of disease. MRI allows for the non-invasive imaging of soft tissue with a superb spatial resolution.

As a useful extension of its diagnostic use, MRI is also proposed for the monitoring of the delivery of bio-active agents such as therapeutic or diagnostic agents. I.e., MRI can not only be used for treatment planning, but also to control local drug delivery under image guidance.

A reference in this respect is Ponce et al., J Natl Cancer Inst 2007; 99: 53-63. Herein a drug, doxorubicin, is taken up in a temperature-sensitive liposome that is solid at normal body temperature and melts at a few degrees higher (41-42° C.). Thus, drug release can be facilitated by applying heat, as this will result in the opening-up of the liposome, whereupon drug release is no longer determined by diffusion (if any) through the liposomal shell. In order to monitor drug release by MRI, a manganese salt is added to the formulation as an MRI contrast agent.

Almost all current MRI scans are based on the imaging of bulk water molecules, which are present at a very high concentration throughout the whole body in all tissues. If the contrast between different tissues is insufficient to obtain clinical information, MRI contrast agents (CAs), such as low molecular weight complexes of gadolinium, are administered. These paramagnetic complexes reduce the longitudinal (T₁) and transverse relaxation times (T₂) of the protons of water molecules. Also manganese ions act as a T₁ contrast agent.

The manganese contrast agent in the aforementioned drug carrier will act upon its exposure to the bulk water molecules, which can be detected by MRI, i.e. it will lead to instantaneous MRI contrast enhancement upon opening up of the liposomal shell above the melt transition temperature of the lipids after the application of heat.

As described, the MRI used in this drug release process is in fact used to monitor the actual release, so as to confirm that the thermo-sensitive liposomes actually work. I.e., it merely provides ex post facto information.

Further, the aforementioned drug delivery is done on the basis of a liposome as a carrier. Liposomes are generally characterized by a lipid bilayer enclosing a cavity. The present invention is in the field of polymersomes, i.e. polymeric vesicles, notably microvesicles and nanovesicles. On the basis of polymersomes, a range of advantages can be achieved as compared to liposomes. Polymersomes are believed to be less prone to macrophage uptake and, hence, are long circulating. Also, as compared to liposomes, polymersomes are more rigid, less dynamic, and more versatile.

### SUMMARY OF THE INVENTION

It would be advantageous to monitor the fate of a drug carrier as of its administration.

In order to better address the aforementioned desire, in one aspect, a Chemical Exchange-dependent Saturation Transfer (CEST) contrast agent for Magnetic Resonance Imaging (MRI) is presented that comprises a polymersome provided with a paramagnetic agent, the polymersome comprising a polymeric shell enclosing a cavity, wherein the cavity comprises a pool of proton analytes, and wherein the shell allows diffusion of the proton analytes.

In another aspect, the polymersome can be a nanoparticle not comprising a cavity.

In another aspect, a drug carrier adapted for localized drug delivery is presented, comprising a contrast agent for CEST MRI that comprises a polymersome, particularly an environment-sensitive polymersome, and a drug.

In a further aspect, a method is presented for the MRI guided delivery of a bio-active agent to a subject, comprising the administration of a drug carrier comprising a polymersome, and being a CEST MRI contrast agent, to said subject, rendering an MR image using the CEST contrast enhancement provided by said administered drug carrier, and allowing the drug carrier to release the bio-active agent.

### DETAILED DESCRIPTION OF THE INVENTION

In a broad sense, the invention can be described with reference to a polymersome-based CEST contrast agent, suitable for use as a drug carrier for localized drug delivery. The suitability of the drug carrier for localized drug delivery can refer to a variety of ways in which a drug carrier loaded with a drug can be triggered to release the drug locally, e.g. by applying a controlled external force or delivering a sufficient amount of energy. This refers, e.g. to environment-sensitive drug carriers that can be triggered to locally release a drug by a change in the environment (e.g. pH in the case of pH-sensitive carriers or applying local heat in the case of thermosensitive carriers). Other methods for localized delivery do not necessarily involve thermosensitive carriers or pH-sensitive carriers, but carriers that can be triggered to release a drug by a method of activation governed by properties other than thermosensitivity or pH-sensitivity, including but not limited to the presence of a gaseous core and/or layers, sensitive to externally applied ultrasound frequency/wavelength and intensity.

### CEST MRI

The invention relates to CEST MRI contrast enhancement. This method serves to generate image contrast by utilizing Chemical Exchange-dependent Saturation Transfer (CEST) from selected, magnetically pre-saturated protons to the bulk water molecules determined by MRI.

CEST in combination with a paramagnetic chemical shift reagent (ParaCEST) is a method in which the magnetization of a pool of paramagnetically shifted protons of a CEST contrast agent is selectively saturated by the application of radio frequency (RF) radiation. The transfer of this saturation to bulk water molecules by proton exchange leads to a reduced amount of excitable water protons in the environment of the CEST contrast agent. Thus a decrease of the bulk water signal intensity is observed, which can be used to create a (negative) contrast enhancement in MRI images.

An approach to obtain a high CEST efficiency is based on utilizing the large number of water molecules of a solution containing a paramagnetic shift reagent (e.g. Na[Tm(dotma)(H₂O)]), wherein “H₄dotma” stands for α,α′,α,″α′″-tetramethyl-1,4,7,19-tetraacetic acid and dotma represents the respective fourfold deprotonated tetraanionic form of the ligand, to provide a pool of protons that are chemically shifted and that, therefore, can selectively be saturated by an RF pulse. If this system is encapsulated in a carrier, here a polymersome, the magnetic saturation can be transferred to the bulk water molecules at the outside of the carriers, which are not chemically shifted. The amount of magnetization transfer and hence the extent of contrast enhancement are determined by the rate of the diffusion of water through the shell of the carrier (i.e. the water exchange rate), as well as by the amount of water within the carrier.

The optimum water exchange rate is directly correlated with the chemical shift difference between the proton pool inside of the carrier and the bulk water outside of the carrier. The paramagnetic shift that is induced on the water molecules inside the polymersomes consists of two main contributions: chemical shift resulting from a direct dipolar interaction between the water molecules and the shift reagent (δ_{dip}), and chemical shift caused by a bulk magnetic susceptibility effect (δ_{bms}). The overall paramagnetic shift is the sum of these two contributions:

δ=δ_{dip}+δ_{bms} (1)

δ_{bms} is zero for spherical particles, but it can be significant for anisotropic particles. The aspherical particles experience a force in a magnetic field, which causes them to align with the magnetic field lines. In the case of liposomes it has been demonstrated that the overall paramagnetic shift can be further increased, if they bear paramagnetic molecules associated with the phospholipid membrane.

A reference on CEST using aspherical liposomes is Terreno, E. et al. Angew. Chem. Int. Ed. 46, 966-968 (2007).

### Polymersomes

The term “polymersomes” is used here to generally indicate nanovesicles or microvesicles comprising a polymeric shell that encloses a cavity. These vesicles are preferably composed of block copolymer amphiphiles. These synthetic amphiphiles have an amphiphilicity similar to that of lipids. By virtue of their amphiphilic nature (having a more hydrophilic head and a more hydrophobic tail), the block copolymers will self-assemble into a head-to-tail and tail-to-head bilayer structure similar to liposomes.

Compared to liposomes, polymersomes have much larger molecular weights, with number average molecular weights typically ranging from 1000 to 100,000, preferably of from 2500 to 50,000 and more preferably of from 5000 to 25000.

The terms “more hydrophilic” and “more hydrophobic” are used in a relative sense. I.e., both can be either hydrophilic or hydrophobic, as long as the difference in polarity between the blocks is sufficient for the formation of the aforementioned polymersomes However, in view of the creation of a cavity in which water can be easily incorporated, it is preferred for the more hydrophilic end of the polymer to be hydrophilic per se.

Further, in view of the use as a drug carrier, it is desired that also hydrophobic drugs can be incorporated into the polymersomes. To this end, it is preferred that the hydrophobic end of the polymer is hydrophobic per se.

The amphiphilic nature of the block copolymers is preferably realized in the form of a block copolymer comprising a block made up of more hydrophilic monomeric units (A) and a block made up of more hydrophobic units (B), the block copolymer having the general structure AₙBₘ, with n and m being integers of from 5 to 5000, preferably 10 to 1000, more preferably 10 to 500. It is also conceivable that one or more further units or blocks are built-in, e.g. a unit C with an intermediate hydrophilicity so as to yield a terpolymer having the general structure AₙCₚBₘ, with n and m being as defined above, and p being an integer of from 5 to 5000, preferably 10 to 1000, more preferably 10 to 500. Any of the blocks can itself be a copolymer, i.e. comprise different monomeric units of the required hydrophilic respectively hydrophobic nature. It is preferred that the blocks themselves are homopolymeric. Any of the blocks, in particular the more hydrophilic block, may bear charges. The number and type of charges may depend on the pH of the environment. Any combination of positive and/or negative charges on any of the blocks is feasible.

In view of the applicability in agents for medical diagnostics and treatment, it is preferred that the polymeric blocks are made of pharmaceutically acceptable polymers. Examples hereof are e.g. polymersomes as disclosed in US 2005/0048110 and polymersomes comprising thermo-responsive block co-polymers as disclosed in WO 2007/075502. Further references to materials for polymersomes include WO 2007081991, WO 2006080849, US 20050003016, US 20050019265, and U.S. Pat. No. 6,835,394.

It is conceivable that a polymersome-like structure can be generated on the basis of a block copolymer, such as a block terpolymer, that intrinsically has the properties of forming a shell enclosing a cavity.

In the present invention, the use of polymersomes facilitates the creation of several advantages.

With respect to the use as a CEST MRI contrast agent, the polymeric nature of the shell leads to the possibility to incorporate a variety of desired units. Thus, e.g., in order to achieve an increased contrast enhancement, the polymer itself can be rendered paramagnetic by the incorporation of metallopolymer units, the enrichment of polymeric units with metal, or both. This refers to e.g. enrichment by including lanthanide-containing lipids into the polymersome structure, or by using lanthanide-containing copolymers. General references regarding metallopolymers are D. Wöhrle, A. D. Pomogailo “Metal Complexes and Metals in Macromolecules” Wiley-VCH: Weinheim, 2003, and R. D. Archer “Inorganic and Organometallic Polymers” Wiley-VCH: New York, 2001. Preferably, the metallopolymer comprises one type or different types of paramagnetic metal ions with a high magnetic moment, such as lanthanide ions. Particularly suitable lanthanides are e.g. gadolinium, terbium, dysprosium, holmium, erbium, thulium, and ytterbium. A reference regarding lanthanide-containing metallopolymers is M. J. Allen, R. T. Raines, L. L. Kiessling, Journal of the American Chemical Society 2006, 128, 6534-6535. The metal ion may be a part of the polymeric backbone or it may be linked to the polymeric chain via a linker connecting the polymer chain to a ligand encapsulating the metal. A reference for suitable encapsulating ligands is P. Caravan, J. J. Ellison, T. J. McMurry, R. B. Lauffer, Chemical Reviews 1999, 99, 2293-2352.

The CEST effect can be tuned by the nature of the blocks of the copolymer and/or the thickness of the polymer layer, since these parameters influence the rate of water exchange across the membrane; E.g. the amphiphilic nature of the polymer can be employed to affect the proton exchange rate through the polymersome. This can generally be done by changing the ratio of lengths of the more hydrophilic and the more hydrophobic blocks. Compared to conventional liposomes, polymersomes are believed to have the advantage of being long circulating, as they are less prone to macrophage uptake.

In accordance with the invention, the use of polymersomes in CEST MRI contrast agents further leads to advantages that can be specifically addressed if the contrast agents are used as a drug carrier. E.g., by virtue of the great versatility of the polymersome structure, one can choose to incorporate both a drug and a paramagnetic agent into the polymersome cavity (and thus have the same distribution of drug and paramagnetic agent), or one can choose to separate the two, and create a different distribution of the drug and the agent, e.g. if the drug is included in the cavity, and the paramagnetic agent is included in the polymer shell. Or, e.g. one can have a drug combination by providing a hydrophilic drug in the cavity, and a hydrophobic drug in the shell.

Polymersomes are semipermeable. In general this refers to the property of the shell to be selectively permeable, sometimes also denoted semi-permeable, or partially or differentially permeable. It indicates a structure that basically is closed in the sense that it is a not fully open wall, and preferably a mostly closed wall, (in this case a shell enclosing a cavity), that allows certain molecules or ions to pass through it by diffusion.

The versatility of polymersomes, i.e. the general freedom to choose the exact chemical structure of the copolymeric blocks, can be used with advantage if the polymers are chosen so as to be biodegradable, to be environment sensitive, or both. This will be explained below with reference to particular advantages for the use as drug carriers.

The present invention judiciously combines the advantages of using CEST MR contrast agents in drug delivery, with desirable properties of polymersomes.

As compared to liposomes, polymersomes are chemically more stable, less leaky, less prone to interfere with biological membranes, and less dynamic due to their lower critical aggregation concentration. These properties result in less opsonisation and longer circulation times.

Polymersomes containing hydrolysable diblock copolymers of polyethylene glycol-polylactic acid have been used as a delivery system for doxorubicin, see e.g. Ahmed, F.; Discher, D. E. Journal of Controlled Release 2004, 96, (1), 37-53. The release of doxorubicin from therewith loaded polymersomes can be triggered by pH. See Ahmed et al., Molecular Pharmaceutics 2006, 3, (3), 340-350. The in vivo stability of polymersomes can be tuned as desired for the specific application.

Though the drug delivery process can be externally induced, the drug delivery process itself can not be imaged in the methods applied before the present invention, which results in an insufficient overall control of the therapeutic intervention. For instance, polymersomes loaded with porphyrin-based fluorophores can be utilized as NIR-emitting probes for in vivo optical imaging in rats. See Ghoroghchian et al. Proceedings of the National Academy of Sciences of the United States of America 2005, 102, (8), 2922-2927.

However, a major disadvantage of optical imaging is the limited penetration depth of light, which currently hampers the translation of optical imaging from animals to humans. The CEST contrast enhancement used in the invention is well suited to conduct human in vivo MR imaging.

The agents of the invention are well suited for an analysis of the spatial distribution of polymersomes loaded with a bioactive compound prior to drug release. Furthermore the intensity of the CEST MR signal scales with the amount of released drug, which allows for quantitative control of the delivered drug dose in vivo. The release of drugs from the polymersome at the diseased site can be triggered by stimuli (e.g. local heating using RF or ultrasound, pH, (enzymatic) hydrolysis of the (biodegradable) polymer chain due to the incorporation of diblock copolymers that are responsive to those).

Other advantages of the invention include that the contrast enhancement can be switched on and off at will. Furthermore, the CEST effect can be tuned by the nature of the (biodegradable) blocks of the copolymer and/or the thickness of the polymer layer, since these parameters influence the rate of water exchange across the membrane. The chemical shift difference between the two proton pools, i.e. the pool within the polymersome and that in its surrounding environment, can be amplified by the aforementioned incorporation of paramagnetic compounds within the polymer bilayer, for instance lanthanide containing lipids or lanthanide containing copolymers. In contrast to liposomes, polymersomes offer the additional advantage that they allow for the incorporation of a well-defined metallopolymer or a metal enriched polymer instead of a single paramagnetic complex per amphiphilic molecule. This is highly beneficial, because the chemical shift difference between the entrapped water and the bulk water scales with the amount of paramagnetic compound.

These various advantages all contribute to the opportunities for image-guided therapy and molecular MR imaging offered by the invention based on polymersomes as CEST contrast agents.

For further background on polymersomes, and the manufacture thereof, reference is made to Antonietti et al., Adv. Mater. 2003, 15, No. 16 and to Soo et al., J. Pol. Sci. Part B: Polymer Physics, Vol. 42, 923-938 (2004).

### Drug Carrier

The drug carrier is to be introduced into the body of a person to be subjected to MRI. This will be e.g. by injection in the blood stream, or by other methods to introduce the carrier into body fluid.

A drug is a chemical substance used in the treatment, cure, prevention, or diagnosis of a disease or disorder, or used to otherwise enhance physical or mental well-being. The guided delivery foreseen with the present invention will mostly be useful for therapeutic agents (i.e. drugs in a strict sense, intended for therapy or prevention of diseases or disorders), but also for agents that are administered for diagnostic purposes. Although other bio-active agents, i.e. those that are not therapeutic or diagnostic, such as functional food ingredients, will not generally be subjected to guided and/or monitored delivery, such could be done using the present invention if desired.

The most optimal use of the invention is attained in the case of targeted therapeutics, i.e. drugs that are intended for targeted delivery, as such delivery will by nature benefit most from the monitoring made available by the invention. This pertains, e.g., to agents in the treatment of tumors to be delivered on site, to agents in the treatment or prevention of cardiovascular disorders, such as atherosclerosis in the coronary arteries, or to antithrombotic agents (e.g. for locally resolving blood cloths) or agents that require passing the blood-brain barrier such as neuromodulators as can be used in the treatment of neural conditions such as epilepsy, Alzheimer's disease, Parkinson's disease, or stroke. Benefits from the guidance and monitoring of targeted drug delivery are also applicable to targeted diagnostic agents. Similarly as with targeted therapeutics, here too cancer is an area where site-specific delivery can be of importance.

Bio-active agents suitable for use in the present invention include biologically active agents including therapeutic drugs, endogenous molecules, and pharmacologically active agents, including antibodies; nutritional molecules; diagnostic agents; and additional contrast agents for imaging. As used herein, an active agent includes pharmacologically acceptable salts of active agents.

The polymersome-based drug carriers of the present invention can comprise either hydrophilic or hydrophobic bioactive agents. A hydrophilic bioactive agent could be encapsulated in the aqueous compartment of the carrier or it could be associated with the more hydrophilic part of the particle shell or its distribution could involve a combination of these options, whereas hydrophobic bioactive agents could be incorporated in hydrophobic domains of the carrier, for instance in the polymersome shell. Nucleic acids, carbohydrates and, in general, proteins and peptides are water soluble or hydrophilic. For instance, bioactive agents which are small molecules, lipids, lipopolysaccharides, polynucleotides and antisense nucleotides (gene therapy agents) are also envisaged. Such biologically active agents which may be incorporated thus include non-peptide, non-protein drugs. It is possible within the scope of the present invention to incorporate drugs of a polymeric nature, but also to incorporate drugs of a relatively small molecular weight of less than 1500 g/mol, or even less than 500 g/mol.

Accordingly, compounds envisaged for use as bioactive agents in the context of the present invention include any compound with therapeutic or prophylactic effects. It can be a compound that affects or participates in tissue growth, cell growth, cell differentiation, a compound that is able to invoke a biological action such as an immune response, or a compound that can play any other role in one or more biological processes.

Relatively small peptides may be referred to by the number of amino acids (e.g. di-, tri-, tetrapeptides). A peptide with a relatively small number of amide bonds may also be called an oligopeptide (up to 50 amino acids), whereas a peptide with a relatively high number (more than 50 amino acids) may be called a polypeptide or protein. In addition to being a polymer of amino acid residues, certain proteins may further be characterized by the so called quaternary structure, a conglomerate of a number of polypeptides that are not necessarily chemically linked by amide bonds but are bonded by forces generally known to the skilled professional, such as electrostatic forces and Vanderwaals forces. The term peptides, proteins or mixtures thereof as used herein is to include all above mentioned possibilities.

Usually, the protein and/or peptide are selected on the basis of its biological activity. Depending on the type of polymer chosen, the product obtainable by the present process is highly suitable for controlled release of proteins and peptides. In a particular embodiment, the protein or peptide is a growth factor.

Other examples of peptides or proteins or entities comprising peptides or proteins which may advantageously be contained in the loaded polymer include, but are not limited to, immunogenic peptides or immunogenic proteins.

Apart from bioactive agents which are water soluble, other water-soluble compounds can be incorporated such as anti-oxidants, ions, chelating agents, dyes, imaging compounds.

Preferred therapeutic agents are in the area of cancer (e.g. antitumor) and cardiovascular disease.

Methods of preparing lipophilic drug derivatives which are suitable for nanoparticle or polymersome formulation are known in the art (see e.g., U.S. Pat. No. 5,534,499 describing covalent attachment of therapeutic agents to a fatty acid chain of a phospho lipid). Drugs in the present invention can also be prodrugs.

The drug may be present in the inner, the outer, or both of the compartments of the carrier, e.g. in the cavity and/or in the shell of the polymersome. The distribution of the drug is independent of the distribution of any other agents comprised in the drug carrier, such as a paramagnetic chemical shift reagent or a paramagnetic agent. A combination of drugs may be used and any of these drugs may be present in the inner, the outer, or both of the compartments of the drug carrier, e.g. in the cavity and/or in the shell of the polymersome.

### Environment-Sensitive Carriers

This refers e.g. to polymersomes having a polymeric shell of which the integrity can be affected by external influences, e.g. heat, pH, polymersomes comprising a gaseous core and/or gaseous layers, polymersomes that are sensitive to externally applied ultrasound frequency/wavelength and intensity. Such polymersomes can be triggered to release a drug (the release of which can be monitored by virtue of the CEST effect), when and where desired by applying the appropriate environmental condition. Furthermore, this can be used to release a drug or a diagnostic agent (and monitor the release) on a site in the body intrinsically having the appropriate condition. As an example of such a condition, reference is made to the commonly decreased pH in tumor cells.

The environment sensitivity can also refer to polymers that are biodegradable. This can be used, e.g., to generate CEST MRI contrast agents, and notably drug carriers based thereon, that have a predetermined life under the circumstances found in a human body. At the end of their lifetime, they will degrade and, as a consequence, the CEST effect vanishes and will thus not appear (thus allowing the indirect determination of the degradation of the drug carrier).

The invention preferably provides for carriers that are thermosensitive. This means that the physical or chemical state of the carrier is dependent on its temperature.

Any thermosensitive carrier that can package a molecule of interest and that is intact at body temperature (i.e. 37° C.) but destroyed at any other, non-body temperature that can be tolerated by a subject may be used. Carriers of the invention include but are not limited to thermosensitive micro- and nanoparticles, thermosensitive polymersomes, thermosensitive nanovesicles and thermosensitive nanospheres, all based on polymers.

Thermosensitive nanovesicles generally have a diameter of up to 100 nm. In the context of this invention, vesicles larger than 100 nm, typically up to 5000 nm, are considered as microvesicles. The word vesicle describes any type of micro- or nanovesicle. Thermosensitive nanospheres include but are not limited to spheres which are no smaller than nanometers. Nanospheres typically do not contain a cavity, i.e. in this embodiment of the invention the CEST effect should be realized purely by chemically shifted protons of the paramagnetic chemical shift agent itself, that is comprised in the nanosphere. This is useful for localizing the nanospheres. For monitoring drug release it will be preferred to use carrier the CEST effect of which is affected by drug release. Hence, polymersomes comprising a cavity are preferred.

Thermosensitive polymersomes include those having a prolonged half-life, e.g. PEGylated polymersomes.

In this description, the semipermeability of the shell generally refers to its ability to allow the MR analyte to pass through it by diffusion. Hence, if the combination of analyte (such as water, or other small molecules comprising protons) and shell (such as a amphiphilic polymer bilayer) is such that the analyte is capable of passing through the shell by diffusion, whereas other molecules, such as the chemical shift agent or the hydrophilic drug, cannot pass the membrane.

References on environment-sensitive carriers having a semipermeable shell, are e.g. U.S. Pat. No. 6,726,925, US 2006/0057192, US 2007/0077230A1 and JP 2006-306794. Further reference is particularly made to Ahmed, F.; Discher, D. E. Journal of Controlled Release 2004, 96, (1), 37-53; to Ahmed, F.; Pakunlu, R. I.; Srinivas, G.; Brannan, A.; Bates, F.; Klein, M. L.; Minko, T.; Discher, D. E. Molecular Pharmaceutics 2006, 3, (3), 340-350; and to Ghoroghchian, P. P.; Frail, P. R.; Susumu, K.; Blessington, D.; Brannan, A. K.; Bates, F. S.; Chance, B.; Hammer, D. A.; Therien, M. J. Proceedings of the National Academy of Sciences of the United States of America 2005, 102, (8), 2922-2927. Based on the description of this invention, the reference to these disclosures will enable the person skilled in the art to execute CEST contrast enhancement using environment-sensitive polymersomes.

Thermosensitive polymersomes for use in the invention ideally retain their structure at about 37° C., i.e. human body temperature, but are destroyed at a higher temperature, preferably only slightly elevated above human body temperature, and preferably also above pyrexic body temperature. Typically about 42° C. is a highly useful temperature for thermally induced (local) drug delivery. Heat can be applied in any physiologically acceptable way, preferably by using a focused energy source capable of inducing highly localized hyperthermia. The energy can be provided through, e.g., microwaves, ultrasound, magnetic induction, infrared or light energy.

Entrapment of a drug or other bio-active agent within polymersomes of the present invention may also be carried out using any conventional method in the art. In preparing polymersome compositions of the present invention, stabilizers such as antioxidants and other additives may be used as long as they do not interfere with the purpose of the invention. Examples include co-polymers of N-isopropylacrylamide (Bioconjug. Chem. 10:412-8 (1999)).

Polymersomes and other potential carriers based on a semipermeable shell enclosing a cavity, will generally be spherical. For use in the invention, it is preferred to render such spherical carriers aspherical. E.g. in the case of polymersomes, this is done by subjecting the polymersomes to a dialysis process against a hypertonic buffer solution, hence a buffer solution with a higher osmolarity compared to the solution at the inside of the polymersomes. The dialysis causes a net diffusion of water from the inside of the polymersomes to the bulk solution. This reduces the total inner volume of the polymersomes. Since the surface area of the polymersomes remains constant, the volume reduction forces the polymersomes to deform and to assume an aspherical shape, such as a disk shape, a cigar shape, or any other aspherical shape.

### The Paramagnetic Chemical Shift Reagent

In the invention, a paramagnetic shift reagent can be comprised in any manner in or on the carrier. It is preferred to have the shift reagent in sufficient interaction with a pool of protons by comprising both the reagent and the pool in the cavity of the carrier.

The paramagnetic chemical shift reagent or reagents can basically be any paramagnetic agent suitable to render the relatively large number of water molecules of a solution or dispersion in which it is contained, into a pool of protons that are chemically shifted regarding their MR resonance frequency, with respect to the surrounding protons of the bulk water molecules. As the polymersomes comprise a shell that fundamentally allows exchange of protons with their direct environment, the saturation caused by a selective RF pulse will be transferred to the environment of the loaded thermosensitive drug carriers. Thus, upon conducting magnetic resonance imaging, the direct environment of the thermosensitive drug carriers will show a decreased signal intensity as compared to other bulk water molecules, and thus allows to detect the direct environment of the contrast agents due to a decreased signal intensity. The paramagnetic chemical shift reagent is to comprise a paramagnetic compound, i.e. any compound having paramagnetic properties. Preferably the paramagnetic compound comprises a paramagnetic metal ions, e.g. metal ions complexed by chelate ligands. Paramagnetic metal ions are known to the skilled person, and do not require elucidation here. E.g., early and late transition metals, explicitly including chromium, manganese, iron, as well as lanthanides, such as gadolinium, europium, dysprosium, holmium, erbium, thulium, ytterbium.

The paramagnetic chemical shift reagent is to comprise a chelating structure capable of strongly binding to the paramagnetic metal and allowing the metal to interact with water, or with another suitable source of protons. With respect to suitable chelating structures, reference is made to P. Caravan et al., Chem. Rev., 99, 2293-2352 (1999). Preferably the water is at least transiently coordinated to the metal of the paramagnetic reagent. With respect to paramagnetic shift mechanisms, reference is made to J. A. Peters et al., Prog. Nucl. Magn. Reson. Spectr., 28, 283-350 (1999). In one embodiment, the chelating structure itself also comprises exchangeable protons, e.g. hydroxy, amine, or amide protons.

Suitably, the paramagnetic chemical shift reagent comprises a lanthanide ion coordinated with a chelating structure, e.g. macrocyclic lanthanide(III) chelates derived from 1,4,7,10-tetraazacyclododecane-1,4,7,10-tetraacetic acid (H₄dota), 1,4,7,10-tetraazacyclododecane-α,α′,α,″a′″-tetramethyl-1,4,7,10-tetraacetic acid (H₄ dotma), and related ligands that allow for an axially coordinated water molecule in the paramagnetic reagent. In this respect reference is made to Aime et al., Angew. Chem. Int. Ed., 44, 5513-5515 (2005). A number of the same, similar or different chelating units may be combined in a dendrimeric or polymeric structure providing dendritic or polymeric chemical shift reagents. A general advantage of using dendritic or polymeric paramagnetic compounds is that high effective concentrations of the paramagnetic metal complex can be achieved, without increasing the osmolarity of the solution as much as it would be the case when using mononuclear paramagnetic compounds. Here reference is made to E. Terreno, A. Barge, L. Beltrami, G. Cravotto, D. D. Castelli, F. Fedeli, B. Jebasingh, S. Aime, Chemical Communications, 2008, 600-602.

Preferably, the paramagnetic chemical shift reagent is water-soluble. Suitable chemical shift reagents are known to the person skilled in the art. The CEST contrast agents do not require any specific chemical shift reagent, as long as the shift reagent and the pool of protons have a sufficient interaction to result in a pool of chemically shifted protons.

Preferably, the paramagnetic shift reagent is a metal complex comprising a metal ion and a ligand that is based on a multidentate chelate ligand. More preferably, the interaction of the chemical shift reagent with the pool of protons is provided in the form of coordination. Thus it is preferred for the metal complex to have at least one coordination site of the metal left open for the coordination of at least one water molecule.

Examples of suitable water-soluble chemical shift reagents are [Ln(hpdo3a)(H₂O)] (1), [Ln(dota)(H₂O)]⁻ (2), [Ln(dotma)(H₂O)]⁻ (3), [Ln(dotam)(H₂O)]³⁺ (4), and [Ln(dtpa)(H₂O)]²⁻ (5), including derivatives thereof and related compounds, with Ln being a lanthanide ion.

Preferably the paramagnetic chemical shift reagent is a lanthanide complex such as in formulae I-5 below:

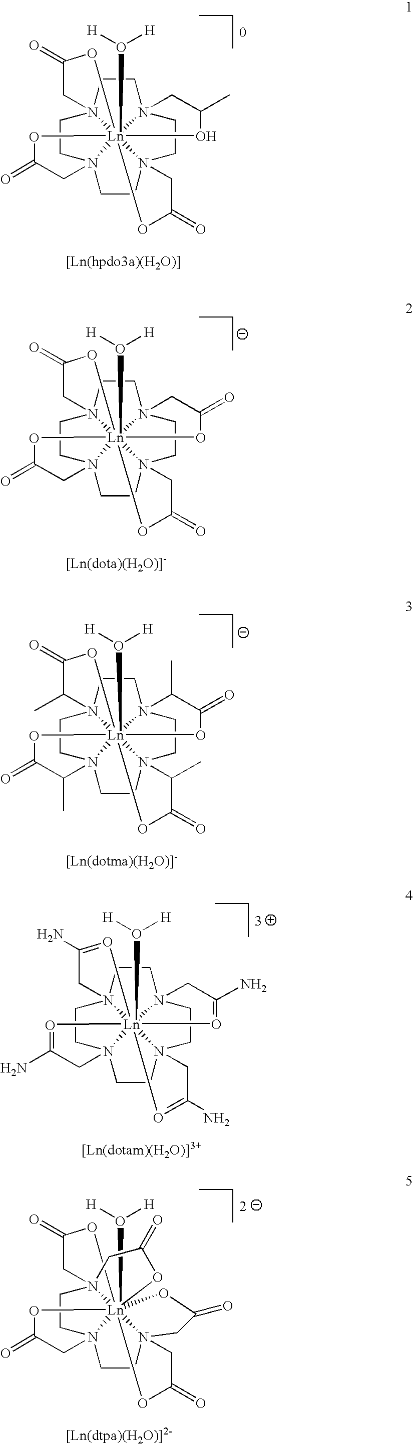

wherein the lanthanide is Eu³⁺, Dy³⁺, Ho³⁺, Er³⁺, Tm³⁺, Yb³⁺, and preferably is Tm³⁺ or Dy³⁺.

The paramagnetic chemical shift reagent is typically comprised in the agent in an amount of from 1 mM to 2000 mM, preferably of from 10 mM to 1000 mM, and more preferably of from 50 mM to 200 mM.

The foregoing metal-containing compounds may be dissolved, emulsified, suspended or in any other form distributed homogeneously or inhomogeneously in the cavity, i.e. the inner compartment of the polymersome. It may alternatively be linked to the outer compartment of the polymersome by at least one covalent or non-covalent bond, or any combination of those. Furthermore the same or at least one different metal-containing compound may be present simultaneously in any of the compartments.

It can be envisaged that the paramagnetic agent and the drug are one and the same, if the drug itself comprises an appropriate metal.

### Further Contrast Enhancement Agents

The contrast agents of the invention may comprise a T₁, T₂ or T₂* reducing agents. In this respect reference is made to Aime et al., Journal of the American Chemical Society, 2007, 129, 2430-2431. In this way an all-in-one concept is realized of T₁, T₂ or T₂* and CEST contrast agents.

The chemical shift difference between the internal and the bulk water protons of the thermosensitive drug carriers can be further enhanced by providing the thermosensitive drug carrier's membrane with a further paramagnetic agent, which is not necessarily a chemical shift reagent. Thus, the orientation of the aspherical carrier in the magnetic field is affected and the aforementioned bulk susceptibility effect is enhanced. The further paramagnetic agent is preferably an amphiphilic compound comprising a lanthanide complex (on the more polar side of the amphilic compound), and having an apolar tail which has a tendency to preferably integrate in and align with the lipid bilayer at the thermosensitive drug carrier's surface based on hydrophobic molecular interactions.

These amphilic paramagnetic complexes can e.g. be:

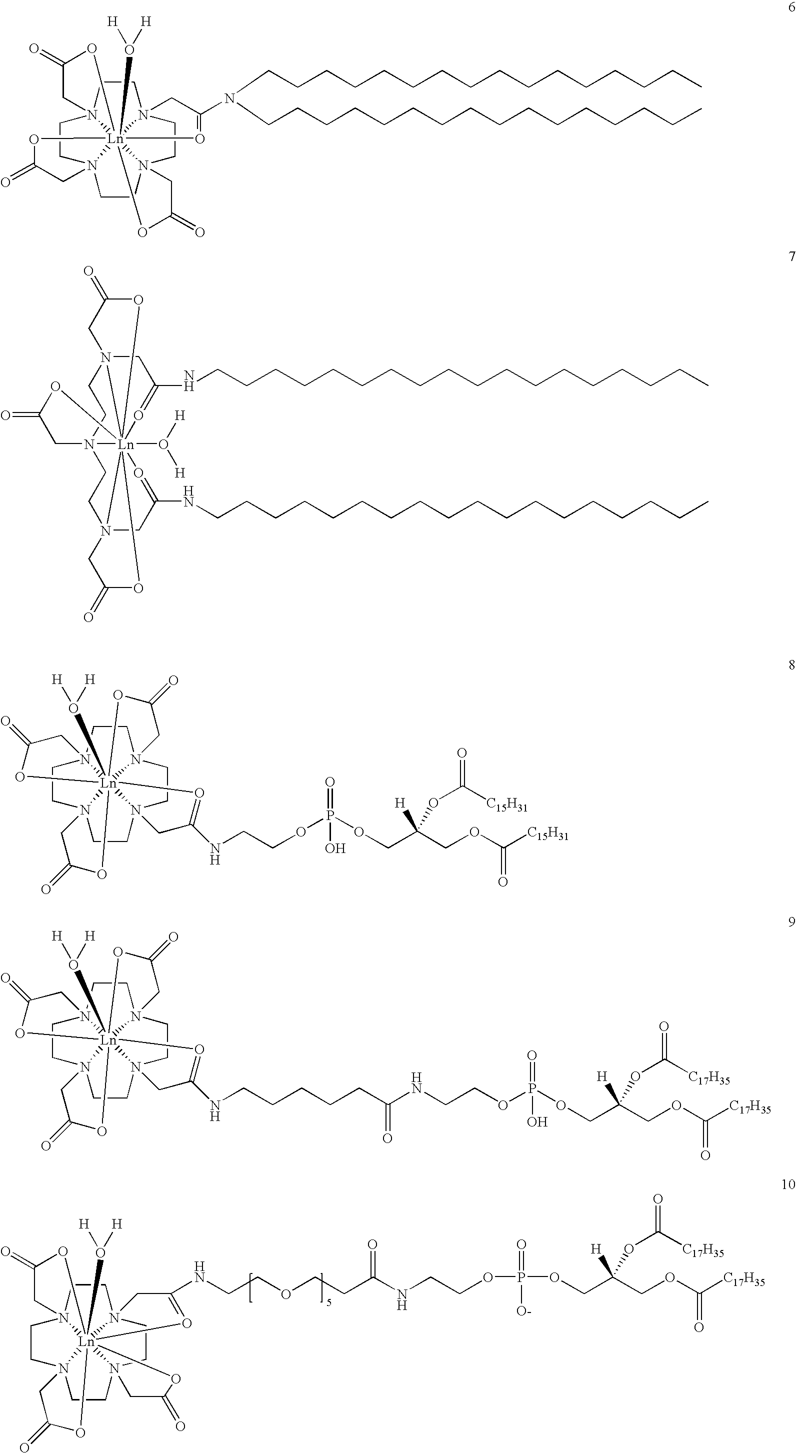

The polymeric nature of the polymersome shell leads to the possibility that the polymer itself can be rendered paramagnetic by the incorporation of metallopolymer units, the enrichment of polymeric units with metal, or both. This refers to e.g. enrichment by including lanthanide-based lipids into the polymersome structure, or by using lanthanide-based copolymers as outlined before.

The lanthanide ion in the optional membrane-associated paramagnetic agent may be identical with or different from the lanthanide within the cavity of the contrast agent.

As is provided according to the invention, the paramagnetic chemical shift reagent can be encapsulated in thermosensitive drug carriers. In this way a pool of water protons is created that has a different chemical shift compared to that of the bulk water surrounding the carriers. The magnetic resonance of these chemically-shifted water protons can be saturated with an RF pulse of a sufficiently narrow band width. Since the water molecules at the inside of the contrast agent are exchanging quickly with the bulk water molecules surrounding the contrast agents, this saturation is transferred to the bulk water.

Hence, when used in practice, at the location of a CEST contrast agent based on thermosensitive drug carriers, the surrounding water (i.e. body fluid in the preferred use in vivo) will be visible as hypointense areas in the CEST-enhanced MR images. With CEST-enhanced MRI, we mean conventional MRI wherein, prior to excitation, the exchangeable-water resonance has been selectively saturated. The RF pulse used for saturation typically has a band-width of several Hertz to several hundred Hertz. The appropriate frequency for the pulse is usually known a priori from phantom or preclinical CEST-MRI studies, but can also be optimized during the actual clinical MRI examination.

Thus, the carriers of the present invention are detectable through MRI at any point in time before the carrier is opened-up. If they additionally comprise T₁ or T₂ contrast agents, also the drug release step upon opening-up of the carrier can be detected (the CEST contrast enhancement will work as long as the shell is closed and exchange of saturated protons can occur through diffusion, the T₁ or T₂ contrast enhancement will display its action when these contrast agents are enabled to interact with bulk water (i.e. body fluid, when said agents are released through the opening up of the shell).

The CEST contrast agents according to the invention can be used in a variety of ways. They can be applied to generate a desired level of MRI contrast in any aqueous environment. Its main use, which is also where the benefits of using thermosensitive drug carriers are enjoyed most, is to generate a local MRI contrast upon in vivo application. This can be by introducing the contrast agents, e.g. by injection into the blood or another body fluid of a living being, preferably a human being, and to perform a CEST contrast-enhanced MRI scan of the body, in whole or in part, of said being. The CEST contrast enhancement of bulk water molecules generated, allows the visibility of spots, such as tumors, where the regular body fluid presence is disturbed. Also, the contrast agents of the invention, in their lipid shell can be provided with disease-specific molecular probes, e.g. by having compounds possessing hydrophobic tail suitable to penetrate into the surface of the carrier (e.g. in the case of a phospholipid surface), wherein the other end of the compounds contains a ligand as desired (i.e. a biochemical ligand for targeted binding).

This allows the contrast agents to preferentially locate at desired or suspect body sites which then can be made visible by MRI. This adds to the suitability of the drug carriers of the invention for localized delivery.

The CEST contrast agents of the invention preferably act on the basis of a pool of protons within the carrier, which exchange with fluid outside of the carrier. This exchange can be done by water-proton transfer, but also by proton transfer from other molecules small enough to pass the shell of polymersomes.

In summary, described in the foregoing are Chemical Exchange-dependent Saturation Transfer (CEST) contrast agents for Magnetic Resonance Imaging (MRI) comprising a polymersome provided with a paramagnetic agent. The polymersome preferably comprises a polymeric shell enclosing a cavity, wherein the cavity comprises a pool of proton analytes, and wherein the shell allows diffusion of the proton analytes. The polymersome-based CEST MRI contrast agents are suitable as drug carriers useful in MRI-guided drug release.

It is to be understood that the invention is not limited to the embodiments and formulae as described hereinbefore. It is also to be understood that in the claims the word “comprising” does not exclude other elements or steps. Where an indefinite or definite article is used when referring to a singular noun e.g. “a” or “an”, “the”, this includes a plural of that noun unless something else is specifically stated.

The invention will be illustrated with reference to the following, non-limiting Example and the accompanying non-limiting Figures.

### Example

Abbreviations used in the Example, to the extent not used hereinbefore: PBD is poly(butadiene), PEO is poly(ethylene oxide), HEPES is (4-(2-hydroxyethyl)-1-piperazine ethane sulfonic acid).

Polymer vesicles with an average diameter of 100-150 nm are formed by a thin film hydration technique coupled with sequential extrusions. In brief, poly(butadiene(1,2-addition)-b-ethylene oxide) with Mₙ (g/mol): PBD(2500)-b-PEO(1300), PD=1.04, and f_{EO}=0.34, is dissolved in CHCl₃. The solvent is gently removed under reduced pressure and a thin polymer film is obtained. The film is hydrated in 20 mM HEPES solution containing 65 mM [Tm(hpdo3a)(H₂O)] and 5 mM carboxyfluorescein. Monodisperse distributions of polymeric vesicles are obtained after sonication at 50° C. for 30 min followed by three freeze-thaw cycles under vacuum using liquid nitrogen at −177° C. and a water bath of 50° C. Subsequently, the dispersion is extruded several times through polycarbonate filters with a pore diameter of 1 μm, 0.4 μm, 0.2 μm, and 0.1 μm. The mean radius of the polymersomes is measured by dynamic light scattering. The shape of the block copolymer vesicles is studied with cryo-transmission electron microscopy. The obtained polymersomes are dialyzed overnight at 4° C. to remove carboxyfluorescein and [Tm(hpdo3a)(H₂O)] not entrapped after hydration of the lipidic film. Dialysis is performed against a buffer with a high ionic strength (20 mM HEPES buffer containing 0.3 M NaCl). The CEST effect of the polymersomes with [Tm(hpdo3a)(H₂O)] in the inside is studied as a function of the saturation frequency offset (FIG. 3) as a function of the saturation RF power (FIG. 4), as a function of time (FIG. 5), and as a function of the concentration Triton X-100 (FIG. 6). For these purposes, a 5 mm glass NMR tube is filled with 0.5 mL of the polymersome formulation. All CEST-MR data are recorded at 7 Tesla on a Bruker Avance 300 NMR spectrometer using a standard continuous-wave irradiation (2 seconds pulse duration; constant amplitude) for selective presaturation of the exchangeable-proton resonance. Typically, 117 individual one-dimensional ¹H-MR spectra are acquired at different values of the presaturation offset frequency (100 Hz intervals till 1000 Hz, then 200 Hz intervals till 4000 Hz, then 400 Hz intervals till 10000 Hz, and 600 Hz intervals till 20200 Hz) centered around the water resonance frequency and stored in a single 2D NMR data set. To reconstruct the Z-spectrum, the water signal of each individual spectrum in the 2D data set is integrated and plotted as a function of the presaturation offset frequency. From the Z-spectrum the CEST effect is determined using equations 1 and 2

CEST effect=(M₀−Mₛ)/M_{∞}×100% (equation 1)

CEST effect=(M₀−Mₛ)/M₀×100% (equation 2)

Here, M_{S} is the bulk-water intensity after an RF saturation pulse applied at a specific offset frequency (e.g. the exchangeable-proton resonance frequency of the contrast agent). M₀ is the bulk-water intensity of the reference experiment, in which the RF saturation pulse is applied symmetrically on the opposite side of the bulk water signal to correct for non-selective saturation, e.g. direct water saturation. M_{∞} is the bulk water intensity at an infinite offset frequency (−200 kHz).

### FIGURES

FIG. 1 is a schematic representation of CEST MRI contrast agents based on polymersomes. The encapsulation of a paramagnetic chemical shift agent, such as [Tm(hpdo3a)(H₂O)], provides a pool of protons that are chemically shifted and that, therefore, can selectively be saturated by an RF pulse (FIG. 1a). The magnetic saturation can be transferred to the bulk water molecules at the outside of the polymersome and hence the extent of contrast enhancement are determined by the rate of the diffusion of water through the polymer bilayer as well as by the amount of water in the lumen of the polymersome (FIG. 1b).

FIG. 2 is a schematic representation polymersomes for image-guided drug delivery. Hydrophilic drugs and imaging agents are encapsulated in the aqueous compartment. Hydrophobic drugs are incorporated in the hydrophobic domains of the polymer bilayer.

FIG. 3 represents Z-spectra of polymersomes loaded with 65 mM [Tm(hpdo3a)(H₂O)] in a buffer solution (20 mM HEPES, pH 7.4) after the application of different presaturation power levels at 310 K and 7 T (left). The CEST effect versus the power level (right). The effect of the chemical exchange of the water in the lumen of the polymersome and the bulk water molecules can be estimated from (M₀−Mₛ)/M_{∞}*100% (equation 1), where Mₛ is the magnitude of the water proton signal after saturation of the water at the inside of the polymersome, M₀ is the intensity of the bulk water proton signal under control irradiation at the opposite frequency offset, and M_{∞} is the magnitude of water proton signal after saturation at an offset frequency of −200 kHz.

FIG. 4 shows the CEST effect of polymersomes loaded with [Tm(hpdo3a)(H₂O)] as a function of the power level at 310 K and 7 T. The effect of the chemical exchange between the water in the lumen of the polymersome and the bulk water molecules can be estimated from (M₀−Mₛ)/M_{∞}×100% (equation 1) and (M₀−Mₛ)/M₀×100% (equation 2), where Mₛ is the magnitude of the water proton signal after saturation of the water at the inside of the polymersomes, M₀ is the intensity of the bulk water proton signal under control irradiation at the opposite frequency offset, and M_{∞} is the magnitude of water proton signal after saturation at an offset frequency of −200 kHz. For comparison with FIG. 3; 44 dB˜9.6 E-6 T; 48 dB˜6.4 E-6 T; 52 dB˜1.2 E-6 T.

FIG. 5 shows the CEST effect of CEST MRI contrast agents based on polymersomes at 323 K and a power level of 44 dB. The CEST effect (according to equation 1) versus the offset frequency (left). The maximum CEST effect as a function of time (right).

FIG. 6 shows the CEST effect of polymersomes loaded with [Tm(hpdo3a)(H₂O)] in the presence of Triton X-100 at 323 K. The CEST effect decreases upon going to higher concentrations of Triton X-100, indicative for the release of [Tm(hpdo3a)(H₂O)] from the polymersome. Moreover, the offset frequency of the chemically shifted intrapolymersomal water decreases as a function of the concentration of Triton X-100 (right).

FIG. 7 displays the normalized fluorescence intensity of polymersomes loaded with 5 mM of carboxyfluorescein and 65 mM of [Tm(hpdo3a)(H₂O)] in a buffer solution (20 mM HEPES, 0.3 N NaCl, pH 7.4) at different temperatures (λₑₓ=488 nm and λₑₘ=512 nm). At 20° C. the normalized fluorescence intensity is constant over time and self-quenching of the fluorescence is observed. Upon addition of Triton X-100 (after 1240 min), the fluorescent probe is released from the polymersome and diluted in the surrounding medium, and the fluorescence intensity increases, so no self-quenching of carboxyfluorescein is observed. At higher temperatures, the intensity of the fluorescence increases over time and at 50° C. saturation of the fluorescence signal is detected after ca. 6 hours. Although the signal appears to be saturated at 50° C., the addition of Triton X-100 results in an additional increase of the fluorescence.

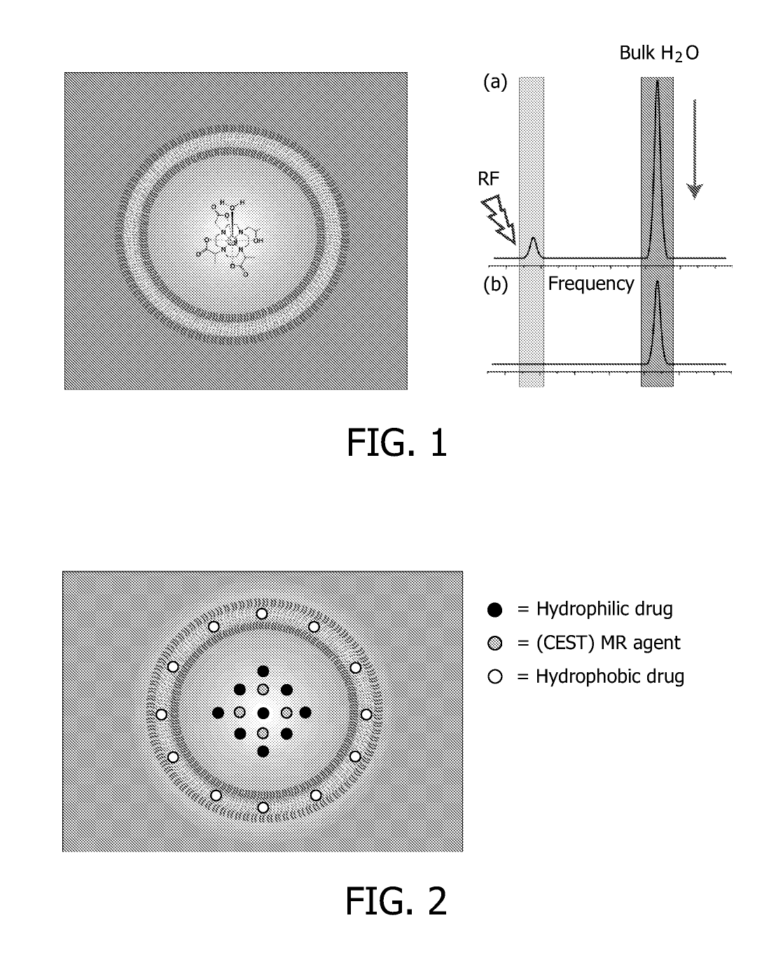

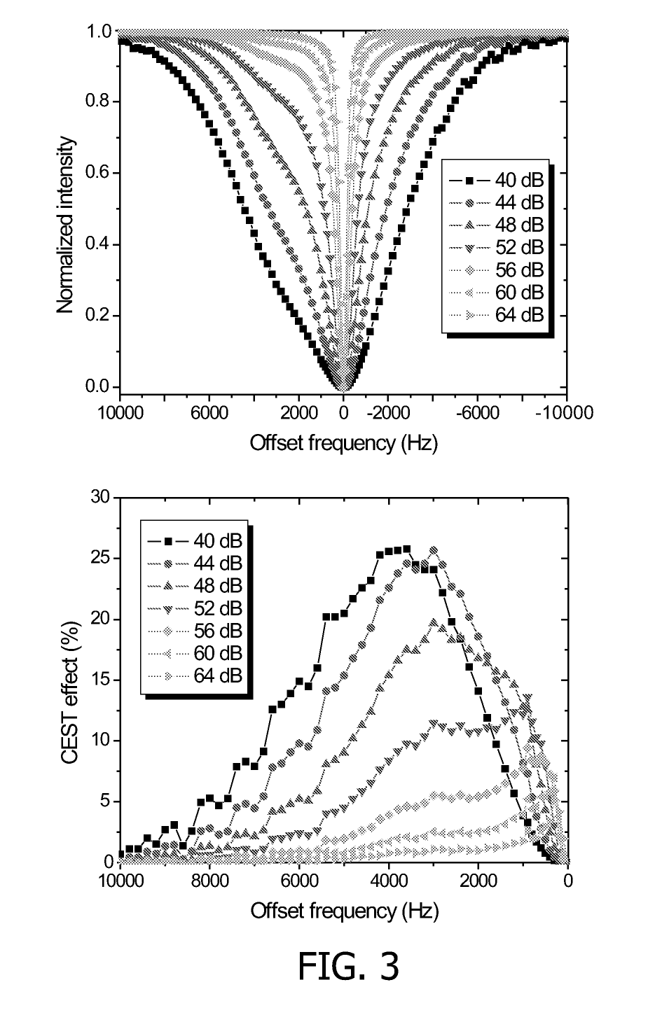

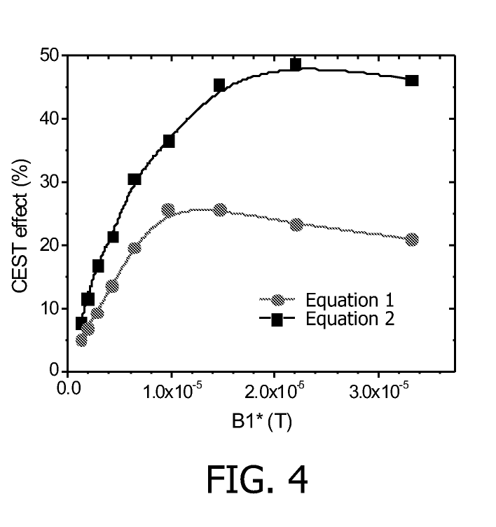

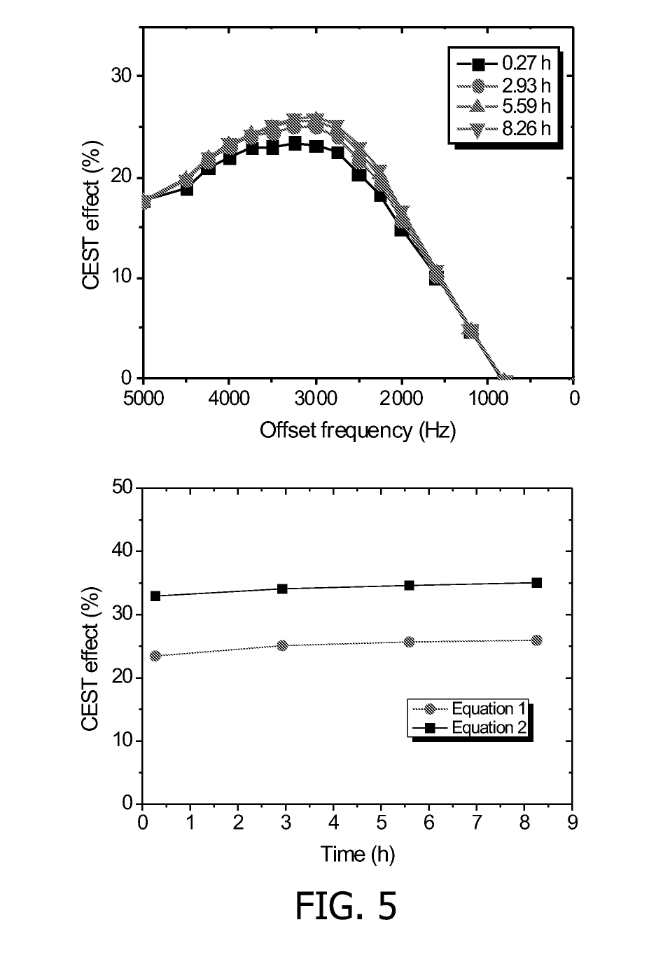

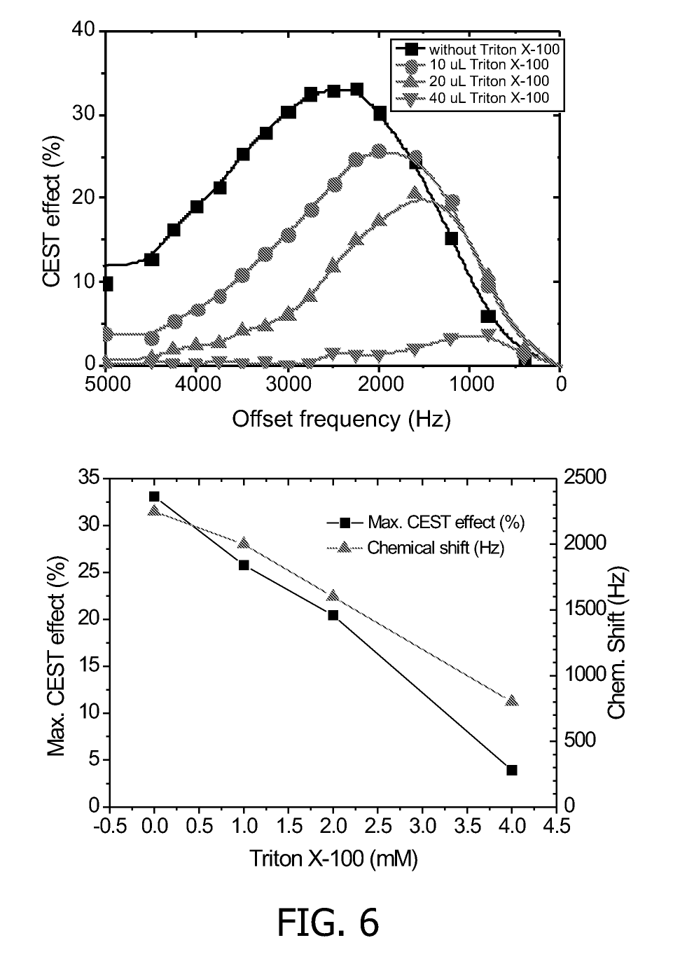

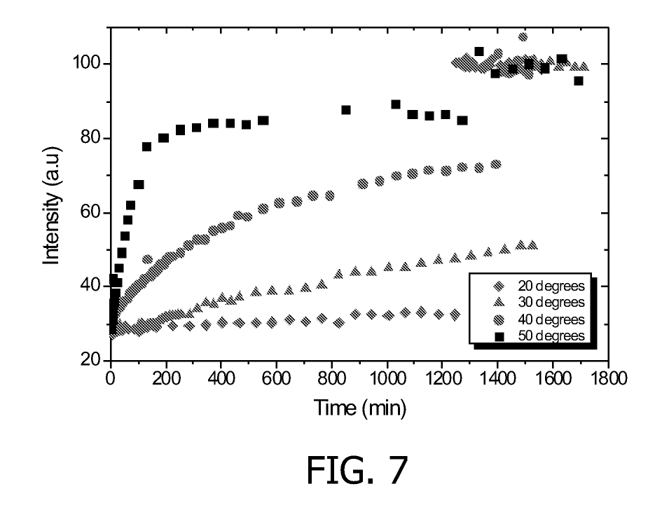

1. A Chemical Exchange-dependent Saturation Transfer (CEST) contrast agent for Magnetic Resonance Imaging (MRI) comprising a polymersome provided with a paramagnetic agent.

2. A contrast agent according to claim 1, wherein the polymersome comprises a polymeric shell enclosing a cavity, wherein the cavity comprises a pool of proton analytes, and wherein the shell allows diffusion of the proton analytes.

3. A contrast agent according to claim 2, wherein the cavity comprises a paramagnetic chemical shift reagent.

4. A contrast agent for CEST MRI according to claim 2, wherein the polymeric shell comprises a bilayer of an amphiphilic polymer.

5. A contrast agent for CEST MRI according to claim 1, having a non-spherical shape.

6. A contrast agent for CEST MRI according to claim 1, wherein the pool of proton analytes comprises water.

7. A contrast agent for CEST MRI according to claim 1, comprising a metal complex having a metal ion and a ligand that is based on a multidentate chelate ligand, as a paramagnetic chemical shift reagent.

8. A contrast agent for CEST MRI according to claim 7, wherein the metal complex has at least one coordination site of the metal left open for the coordination of at least one water molecule.

9. A contrast agent for CEST MRI according to claim 1, comprising a paramagnetic agent in or at its outer surface.

10. A contrast agent for CEST MRI according to claim 1, wherein the polymeric shell comprises a plurality of metal-containing units selected from metallopolymer units, metal-enriched units, and mixtures thereof.

11. A contrast agent for CEST MRI according to claim 2, wherein the polymeric shell is environment-sensitive.

12. A drug carrier adapted for localized drug delivery, comprising a contrast agent for CEST MRI according to claim 11, and a drug.

13. A method for the MRI guided delivery of a bio-active agent to a subject, comprising the administration of a drug carrier according to claim 12 to said subject, rendering an MR image using the CEST contrast enhancement provided by said administered drug carrier, and allowing the drug carrier to release the bio-active agent.

14. The use of a CEST MRI contrast agent according to claim 1 as a drug carrier.

15. A use according to claim 14, wherein the polymersome has a non-spherical shape and does not comprise a paramagnetic shift reagent substantially interacting with the analytes.
